(19) United States
(12) Patent Application Publication
(10) Pub. No.: US 2024/0016143 A1
(43) Pub. Date: Jan. 18, 2024

(54) OPEN CELL HYDROGEL NETWORKS AND METHODS FOR MAKING AND USING THE SAME

(71) Applicant: University of Florida Research Foundation, Inc., Gainesville, FL (US)

(72) Inventors: William R. NIEDERHAUSER, Gainesville, FL (US); Charles SAWYER, Gainesville, FL (US); Wallace G. SAWYER, Gainesville, FL (US); William W. SAWYER, Gainesville, FL (US)

(21) Appl. No.: 18/250,260

(22) PCT Filed: Oct. 29, 2021
(86) PCT No.: PCT/US2021/072104

§ 371 Date: Apr. 24, 2023

Related U.S. Application Data
(60) Provisional application No. 63/107,202, filed on Oct. 29, 2020.

(51) Int. Cl. A01N 25/04; A01N 25/16; A01N 25/10; A01P 19/00

(52) U.S. Cl. CPC A01N 25/04; A01N 25/16; A01N 25/10; A01P 19/00

(57) ABSTRACT

In one aspect, the disclosure relates to open cell hydrogel networks, methods of making the same and baits formed from the same, methods of loading the same with attractant molecules and compositions, and methods of catching fish and/or other aquatic organisms using the same. The open cell hydrogel networks disclosed herein can be configured to have different properties based on the specific polymers used for synthesis as well as any initiators, catalysts, and/or crosslinkers, thus allowing the fabrication of suitable baits for a variety of target species. In some aspects, the open cell hydrogel networks can be used as a filter or drug delivery device.

### CROSS REFERENCE TO RELATED APPLICATIONS

This application claims the benefit of U.S. Provisional Application No. 63/107,202 filed on Oct. 29, 2020, which is incorporated herein by reference in its entirety.

### BACKGROUND

Hydrogels are three-dimensional crosslinked polymer networks with the capability of swelling due to uptake of large amounts of water. Polymeric hydrogels have been used in tissue engineering, sensing, drug delivery, cell culture, would dressings, disposable diapers, adhesives, contact lenses, and even electricity generation and explosives; many are responsive to temperature, pH, salt concentration, light, and/or other environmental factors. Many hydrogels have low permeability for structural and chemical reasons and are therefore slow to release therapeutic and other compounds. Sponge-like structures formed from non-hydrogel materials (e.g., cellulose, melamine, urethane), on the other hand, can also take up and release compounds but do so too quickly for use in delivery of drugs and other compounds to aqueous solutions such as, for example, bodies of water, body cavities and/or surfaces of human or animal subjects, and the like. Materials that are intermediate in permeability between hydrogels and sponges could be useful for sustained delivery of water-soluble compounds and compositions; however, experimentally and industrially, this space remains largely unexplored.

Numerous fish attractants are commercially available, but effective release of the attractants remains a problem. Some lures can be coated with attractants; however, water-soluble attractants applied to lure surfaces quickly disperse into the surrounding water, becoming less concentrated and even ineffective in a short period of time.

Various devices for distributing fish attractants have been proposed. These often involve exterior housings having one or more openings for dispersal of the attractants. These devices can be expensive and may be made from non-environmentally benign and/or non-biocompatible materials including hard plastics and the like. These devices further do not have the feel or texture associated with the tissues of organisms on which fish are motivated to feed. Some devices may only operate at a certain pH or electrolyte concentration; for example, they may function in saltwater but not freshwater, or only at certain pH levels.

What is needed is a material that can provide a controlled release over time of various water-soluble molecules and compositions including, but not limited to, attractant molecules and compositions for fish and other aquatic organisms, pharmaceutical compositions for human and/or animal subjects, and the like. The material would be biocompatible and/or biodegradable, would be inexpensive to produce, would be capable of uptake of large concentrations of attractants or pharmaceuticals, and would ideally mimic the feel of living tissue, both to entice aquatic organisms and for patient comfort. The material would additionally function in water of all salinity and pH levels. These needs and other needs are satisfied by the present disclosure.

### SUMMARY

In accordance with the purpose(s) of the present disclosure, as embodied and broadly described herein, the disclosure, in one aspect, relates to open cell hydrogel networks, methods of making the same and baits formed from the same, methods of loading the same with attractant molecules and compositions, and methods of catching fish and/or other aquatic organisms using the same. The open cell hydrogel networks disclosed herein can be configured to have different properties based on the specific polymers used for synthesis as well as any initiators, catalysts, and/or crosslinkers, thus allowing the fabrication of suitable baits for a variety of target species. In some aspects, the open cell hydrogel networks can be used as a filter or drug delivery device.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows an open cell hydrogel network according to the present disclosure formed into a bait with attached hook and line.

FIG. 1B shows a first exemplary method of loading attractants, scents, small molecules, proteins, and/or amino acids into a bait, i.e., by applying a concentrated aqueous solution thereof to the bait.

FIG. 1C shows a second exemplary method of loading attractants, scents, small molecules, proteins, and/or amino acids into a bait, i.e., by soaking the bait in a concentrated aqueous solution thereof.

FIG. 1D is a schematic of a loaded open cell hydrogel network bait in an aqueous environment, where attractants, scents, small molecules, proteins, and/or amino acids are released from the bait into the surrounding environment.

FIG. 2 is a schematic representing the internal structure of an open cell hydrogel network including both hydrogel aggregated nodes providing slow release of loaded molecules and open spaces in the hydrogel cell network, providing rapid release of loaded molecules.

FIG. 3 is a scanning electron micrograph of the disclosed hierarchical open cell hydrogel network structure showing aggregates, short connective fibrils and junctions, and contiguous free space for flow.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a polymer,” “a catalyst,” or “an attractant molecule,” includes, but is not limited to, mixtures or combinations of two or more such polymers, catalysts, or attractant molecules, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y”’, where ‘x’ and ‘y’ are numerical values, includes “about ‘x’ to about ‘y”’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a polymer refers to an amount that is sufficient to achieve the desired improvement in the property modulated by the formulation component, e.g. achieving the desired level of permeability and/or desired pore size for the polymeric materials and/or open cell hydrogel networks disclosed herein. The specific level in terms of wt % in a composition required as an effective amount will depend upon a variety of factors including the amount and type of polymer, amount and type of initiator, catalyst, and/or cross-linker, amount and type of attractant molecules and/or compositions to be loaded into the hydrogel, and the target fish or other organism to be lured.

As used herein, the terms “optional” or “optionally” means that the subsequently described event or circumstance can or cannot occur, and that the description includes instances where said event or circumstance occurs and instances where it does not.

As used herein, “hydrogel” refers to a network of crosslinked, hydrophilic polymer chains. A hydrogel network does not dissolve in the presence of a high concentration of water. In one aspect, hydrogels can absorb up to or over 90% water. Hydrogels can be biocompatible and may be made from natural or synthetic polymers, or combinations thereof. In one aspect, disclosed herein are permeable hydrogels that can be shaped into baits and used to release attractant molecules and/or compositions into water to attract fish and/or other aquatic organisms.

A “cross-linker” or “cross-linking agent” refers to a chemical species that adds chemical bonds between different chains of atoms in a polymer such as a hydrogel. A cross-linker may affect elasticity, viscosity, glass transition temperature, melting temperature, absorbency, and solubility of hydrogels, as well as combinations thereof. In one aspect, N,N′-methylenebisacrylamide (BIS) can be used as the cross-linker for the hydrogels disclosed herein. In another aspect, cross-linked hydrogels can be produced by chain-growth polymerization including free-radical polymerization, solution polymerization, suspension polymerization, photopolymerization, irradiation polymerization, step-growth polymerization, or any combination thereof. Exemplary cross-linkers are provided below.

As used herein, an “initiator” is a chemical species that reacts with a monomer to form an intermediate, wherein the intermediate can link successively with other monomers to form a polymer. In one aspect, an initiator can be a thermal initiator, a photoinitiator, γ radiation, electron beam, a chemical initiator, or a combination thereof. In one aspect, for the open cell hydrogel networks disclosed herein, ammonium persulfate (APS) is a useful initiator. In another aspect, the initiator is a source of free radicals for the polymerization reaction to form the disclosed open cell hydrogel networks. Exemplary initiators are provided below.

A “catalyst” as used herein is a compound that increases the rate of a chemical reaction. In some aspects, catalysts are useful for making the open cell hydrogel networks disclosed herein. In another aspect, the catalyst can be a free radical stabilizer. In one aspect, the catalyst used herein can be tetramethylethylenediamine (TEMED) or another catalyst. In some aspects, a “catalyst” can also be referred to as an “activator.”

The phrase “attractant molecules and compositions” as used herein refers to scents, attractants, small molecules, proteins, amino acids, or any combination thereof that can be applied to the disclosed open cell hydrogel networks by any method described herein for the purpose of attracting fish, crustaceans, and/or other aquatic life to the baits disclosed herein. In one aspect, the attractant molecules and compositions can be water-soluble.

As used herein, “solvation” refers to the process by which solvent molecules interact with solute molecules. The “solvation limit” is reached when there are no more sites for interaction available on the solute molecules and excess solvent is free in solution. In one aspect, the open cell hydrogel networks disclosed herein form when their monomeric components are polymerized with excess water (e.g., above the solvation limit of the monomeric components).

In one aspect, the open cell hydrogel networks herein include a plurality of polymeric “nodes.” Nodes as used herein are local areas of polymerization having a mesh size of from about 10 nm to about 100 nm. In some aspects, the nodes can aggregate to form “aggregated nodes” having an average size of from about 10 nm to about 500 μm. Between the nodes and/or aggregated nodes are thinner polymeric “fibrils” connecting the nodes. In one aspect, the nodes and aggregated nodes provide for a slow release of attractant molecules and compositions. In another aspect, the nodes are typically spherical in shape.

As used herein, a “fibril” is a thin element connecting polymeric nodes. In a further aspect, a fibril has a diameter significantly less than the average diameter of a node as disclosed herein. In a still further aspect, the length of fibrils can vary based on polymerization conditions, particular polymer identities, and the like. In one aspect, fibrils can impart stability, tensile strength, and other chemical and mechanical properties to the open cell hydrogel networks. In another aspect, the fibrils have an irregular arrangement and randomly connect through nodes.

In another aspect, surrounding the nodes, hydrogel nodes, and fibrils or other attachments between and among nodes is an “open cell network” having a single characteristic or smallest dimension of between about 100 nm and about 5 mm and a tunable permeability of from about 1×10⁻¹⁵ m² to about 1×10⁻⁴ m². In one aspect, the open cell network provides for a rapid release of attractant molecules and compositions. In another aspect, the open cell network has a contiguous connected fluid space. In another aspect, the open cell network is fibrillar with granular aggregates connected together by direct contacts or with fibrils as described herein.

“Permeability” as used herein refers to the ability of a porous material to allow fluids to pass through. Permeability is controlled by a number of factors including porosity of the material as well as shape and connectedness of pores. In one aspect, permeability of the open cell hydrogel networks disclosed herein can be roughly estimated as the square of the diameter of the pores or characteristic dimension of the open cell network.

As used herein, “biocompatible” refers to a material that does not damage cells, tissues, or organs with which it comes in contact. In one aspect, biocompatible materials do not cause allergic reactions, irritation, or sensitivity. In one aspect, the open cell hydrogel networks disclosed herein are biocompatible.

“Biodegradable” refers to an object or composition that can be broken down by microorganisms and/or by the digestive systems of multicellular organisms into its component parts including, but not limited to, oligomers and/or monomers of polymeric components of the object or composition. In one aspect, the open cell hydrogel networks disclosed herein can optionally be biodegradable.

Unless otherwise specified, temperatures referred to herein are based on atmospheric pressure (i.e. one atmosphere).

### Method for Preparing Open Cell Hydrogel Networks

In one aspect, disclosed herein is a method for synthesizing permeable open cell hydrogel networks. In another aspect, stock solutions can be individually prepared for each of a cross-linking agent, an initiator, and/or a catalyst. In some aspects, a polymer stock solution can also be prepared. In other aspects, the polymer is provided in liquid form and used neat.

In one aspect, the method includes the steps of:
a. admixing a crosslinker, an initiator, and a monomer in water to form a first solution; and
b. admixing the first solution with a catalyst to polymerize the monomer to form the hydrogel;
wherein water is present in an amount greater than the solvation limit of the monomer.

In one aspect, the polymers selected for inclusion in the open cell hydrogel networks disclosed herein can be inert or charged and are compatible with the pH and solutes found in the body of water in which they will be used. In one aspect, the open cell hydrogel networks are biodegradable. In an alternative aspect, the open cell hydrogel networks are not biodegradable.

In one aspect, one or more of the solutions of crosslinker, initiator, monomer, and/or catalyst can be degassed prior to performing the method.

Without wishing to be bound by theory, polymerizing the monomer with an excess amount of water (i.e., above the solvation limit of the monomer) results in the formation of the aggregated node structure, wherein the nodes are connected by fibrils and surrounded by an open cell network. In one aspect, no convection occurs in the gel; thus, pressure and current are responsible for elution of any compounds taken up by the gel. In some aspects, including a high amount of catalyst increases polymer nucleation as well as polymerization speed, trapping the hydrogel in the node/open cell network structure disclosed herein. In one aspect, adjustment of polymerization kinetics can be used to fine-tune the size of nodes and pores in the open cell hydrogel networks.

In one aspect, the monomer can include hydroxyethylmethacrylate (HEMA), a polyethylene glycol (PEG) monomer, a polypropylene oxide (PPO) monomer, lactic acid, glycolic acid, N-isopropylacrylamide (NIPAM), caprolactone, R-3-hydroxybutyrate, vinyl acetate, ethylene, acrylamide, methacrylamide, dimethacrylamide, acrylate, methacrylate, acrylic acid, methacrylic acid, C₂-C₈ alkyl acrylic acids, vinyl ester, vinyl amide, vinyl amine, poly(ethylene glycol) diacrylate (PEGDA), N-vinylpyrrolidone, hydroxyethyl acrylate, hydroxypropyl acrylate, trimethylammonium 2-hydroxypropylmethacrylate, dimethylaminoethyl methacrylate (DMAEMA), dimethylaminoethylmethacrylamide, allyl alcohol, vinylpyridine, glycerol methacrylate, N-(1,1-dimethyl-3-oxobutyl)acrylamide, or any combination thereof. In any of these aspects, upon polymerization, the polymers can form a structure including nodes and/or aggregated nodes connected by fibrils and surrounded by an open network. In one aspect, when the polymer is or includes HEMA and is polymerized, a suitable hierarchical microstructure for a soft bait for fishing can be produced.

In some aspects, following polymerization, at least one functional group can be removed from the hydrogel by base-catalyzed hydrolysis. Further in this aspect, the at least one functional group can be an acetate group.

In another aspect, the cross-linking agent can be N,N′-methylenebisacrylamide (BIS) or another cross-linking agent including, but not limited to, sodium borate/boric acid, glyoxal, silane, oxidized dextrins, glutaraldehyde, epichlorohydrin, endogen polyamine spermidine, oxidized alginate, zinc, borax, and/or ethylene glycol dimethacrylate (EGDMA). In one aspect, the cross-linking agent is from about 0.1% (w/v) to about 5% (w/v) in water. In another aspect, when the cross-linking agent is BIS, the BIS can be about 2% (w/v) in water.

In one aspect, the initiator can be ammonium persulfate, potassium persulfate, sodium metabisulfite, azoisobutyronotrile (AIBN), potassiumperoxodisulfate, dibenzoyl peroxide, hydrogen peroxide, sodium percarbonate, benzoin methyl ether, 1-hydroxycyclohexylphenylketone, or any combination thereof, ultraviolet light in the presence of a radical and/or cationic photoinitiator (e.g. benzoin derivatives, acetophenone derivatives, camphorquinone, thioxanthone, benzophenone, or the like), heat, or another initiator. Further in this aspect, the APS can be about 10% (w/v) in water.

In still another aspect, the catalyst can be tetramethylethylenediamine (TEMED) or another catalyst. Further in this aspect, the TEMED can be about 10% (w/v) in water. Suitable catalysts, initiators, and cross-linking agents can be chosen based on the polymer or polymer system used in the process disclosed herein. In one aspect, these components can be mixed in any order. Exemplary methods for preparing the open cell hydrogel networks disclosed herein are provided in the Examples.

In one aspect, the monomer can be HEMA, the crosslinker can be BIS, the initiator can be ammonium persulfate, and the catalyst can be TEMED.

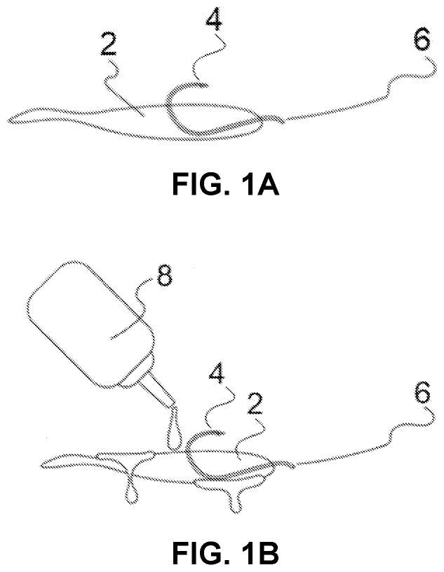

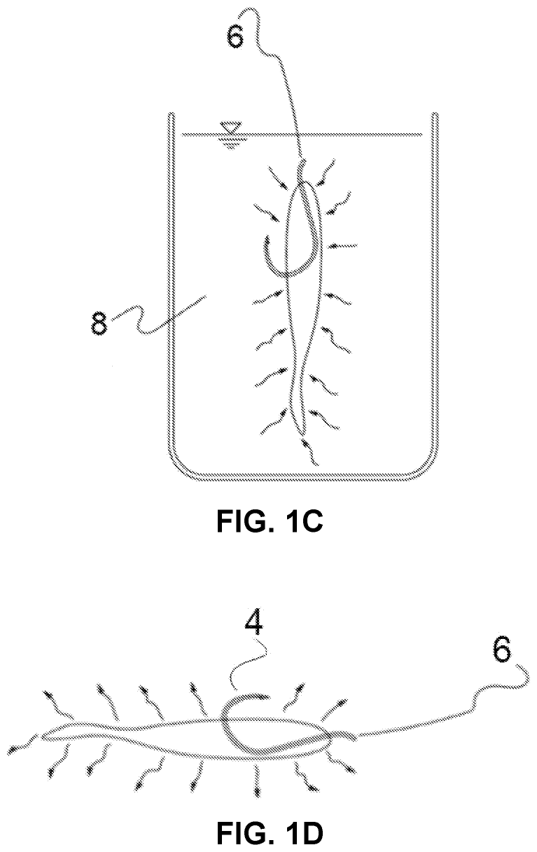

In any of these aspects, the hydrogel can be polymerized in aqueous solution far above the equilibrium water content for a given polymer concentration with associated crosslinking molecules. In one aspect, aqueous BIS and APS can be admixed with liquid HEMA and water and polymerization can be initiated with the addition of TEMED. In some aspects, the mixture containing the initiator, cross-linking agent, catalyst, polymer, and water can be poured into a bait-shaped mold or cut into a bait shape after polymerization has been completed. An exemplary bait 2 according to the present disclosure is shown in FIG. 1A. In some aspects bait 2 can include a hook 4 and/or a line or lead 6.

In a further aspect, the polymers are capable of absorbing water or another solvent and swelling. In a still further aspect, the open network can further fill with water or another solvent.

In any of these aspects, the water or other solvent can include one or more solutes such as, for example, one or more scents, attractants, small molecules, proteins, amino acids, or any combination thereof.

Articles Formed from the Open Cell Hydrogel Networks

In one aspect, disclosed herein is an article including a polymeric material, wherein the article is suited to extended periods of immersion in an aqueous environment and wherein the article is permeable to small molecules, wherein small molecules contained within the article can be released from the article into the aqueous environment. In a further aspect, the polymeric material can be an open cell hydrogel network having a plurality of nodes attached to one another, wherein at least a portion of the nodes cluster to form a plurality of hydrogel nodes, and wherein the hydrogel includes an open cell network.

In a further aspect, the article can be formed into a bait. Further in this aspect, the small molecules to which the article is permeable can be scents, flavors, or other attractant compositions useful for attracting fish and/or other aquatic organisms. Examples of such molecules are disclosed below.

### Hydrogel Structure

In one aspect, the open cell hydrogel networks disclosed herein include both hydrogel nodes and an open network. In a further aspect, the individual hydrogel nodes have a polymer mesh size of from about 10 nm to about 100 nm, or of about 10, 15, 20, 25, 30, 35, 40, 45, 50, 55, 60, 65, 70, 75, 80, 85, 90, 95, or about 100 nm, or a combination of any of the foregoing values, or a range encompassing any of the foregoing values. In another aspect, the aggregated hydrogel nodes have a characteristic size of from about 10 nm to about 500 μm, or of about 10, 25, 50, 100, 200, 300, 400, 500, 600, 700, 800, or about 900 nm, or about 1, 50, 100, 200, 300, 400, or about 500 μm, or a combination of any of the foregoing values, or a range encompassing any of the foregoing values.

In another aspect, the open network of the hydrogels surrounds the hydrogel nodes and has a pore size or characteristic dimension of from about 10 nm to about 1 mm, or of from about 100 nm to about 500 μm, or of from about 1 μm to about 100 μm, or of about 10, 20, 30, 40, 50, 60, 70, 80, 90, 100, 200, 300, 400, 500, 600, 700, 800, or about 900 nm, or about 1, 50, 100, 200, 300, 400, 500, 600, 700, 800, 900, or about 1000 μm, or a combination of any of the foregoing values, or a range encompassing any of the foregoing values. In still another aspect, the open network has a permeability of from about 1×10⁻¹⁵ m² to about 1×10⁻⁴ m², of from about 1×10⁻¹² m² to about 1×10⁻⁸ m², or of about 1×10⁻¹⁵, 1×10⁻¹⁴, 1×10⁻¹³, 1×10⁻¹², 1×10⁻¹, 1×10⁻¹⁰, 1×10⁻⁹, 1×10⁻⁸, 1×10⁻⁹, 1×10⁻⁶, 1×10⁻⁵, 1×10⁻⁴ m², or a combination of any of the foregoing values, or a range encompassing any of the foregoing values. In one aspect, the permeability is from about 5.45×10⁻¹² to about 2.75×10⁻¹². In another aspect, permeability can decrease with increasing polymer concentration.

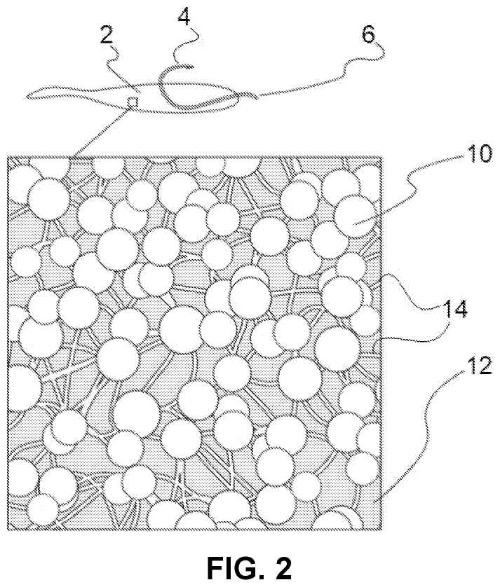

In any of these aspects, the hydrogel nodes can be connected by polymeric and/or hydrogel fibrils or other local attachments. In some aspects, the polymeric and/or hydrogel fibrils pass through the spaces of the open network. A schematic of the hydrogel network is presented in FIG. 2: hydrogel bait 2 having optional hook 4 and line or lead 6 is composed of a hydrogel having hydrogel nodes 10, fibrils 14 connecting the nodes, and open network 12 surrounding the nodes and fibrils.

In one aspect, when the open cell hydrogel networks are equilibrated in water, they can become swollen. In one aspect, the swollen gels can have from about 20% to about 90% water by mass, or about 20, 25, 30, 35, 40, 45, 50, 55, 60, 65, 70, 75, 80, 85, or about 90% water by mass, or a combination of any of the foregoing values, or a range encompassing any of the foregoing values. In another aspect, the open cell hydrogel networks can contain from about 10% to about 30% polymer by mass, or about 10, 12, 14, 16, 18, 20, 22, 24, 26, 28, or about 30% polymer by mass, or a combination of any of the foregoing values, or a range encompassing any of the foregoing values. In one aspect, the open cell hydrogel networks contain about 20% polymer by mass. In another aspect, without wishing to be bound by theory, when water concentration is low, the open cell hydrogel networks are relatively impermeable and loaded compounds may not be released from the hydrogel. Thus, further in this aspect, a high water concentration during and after polymerization is important for proper release of attractant molecules, filtered molecules, pharmaceutical compositions, or any other compound or composition with which the open cell hydrogel networks are loaded.

In another aspect, the hydrogel can have a modulus of from about 10 kPa to about 100 MPa, or of from about 10 kPa to about 1 MPa, or of about 10, 50, 100, 200, 300, 400, 500, 600, 700, 800, or 900 kPa, or of about 1, 5, 10, 20, 30, 40, 50, 60, 70, 80, 90, or about 100 MPa, or a combination of any of the foregoing values, or a range encompassing any of the foregoing values.

In one aspect, these properties can be tuned based on the type of target organism, water temperature at the intended time of use, salt and/or other solute concentrations, and the like. In a further aspect, adjustment of properties can provide for fast release, slow release, or sustained release of the solutes over time.

In one aspect, the hydrogel can be charged. In another aspect, the hydrogel can be neutral. In one aspect, the hydrogel can be biodegradable. In another aspect, the hydrogel is not biodegradable.

Methods of Loading Components into Open Cell Hydrogel Networks

In one aspect, various compounds can be loaded into the open cell hydrogel networks. In a further aspect, the open cell hydrogel networks can be formed as baits, with or without hooks and connecting lines or leaders. Further in this aspect, the compounds can include molecules and compositions that can attract fish, crustaceans, and/or other aquatic organisms. In one aspect, the compounds can be selected from scents, attractants, small molecules, proteins, amino acids, and combinations thereof.

In one aspect, the bait can additionally include a filler. In some aspects, the filler can be a mesh, a matrix, a plurality of fibers, or a combination thereof. In another aspect, the filler can be cellulose, cotton, polyethylene, polypropylene, nylon, melamine foam, a metal, or any combination thereof. In one aspect, the bait can form an outer layer around the filler. In an alternative aspect, the filler can be distributed throughout the bait.

In one aspect, the bait further includes a preservative, an antibacterial agent, an antimicrobial agent, an antioxidant, or any combination thereof. In one aspect, the bait can include at least one pigment. In a further aspect, the pigment can be a solid pigment present at the time of polymerization. In another aspect, the pigment can be a chromophore chemically bound to the hydrogel, to a monomer used to form the hydrogel, to a filler used in the construction of the bait, or a combination thereof.

In another aspect, the bait can be formed into the shape of an aquatic organism, an insect, a worm, or fish eggs.

In any of these aspects, prior to or after forming the open cell hydrogel networks into baits, the open cell hydrogel networks can be loaded with one or more attractant molecules or compositions. In a further aspect, the attractant molecules and/or compositions can be aqueous or water-soluble. In a still further aspect, the attractant molecules and/or compositions can be applied to the open cell hydrogel networks by pouring, dripping, spraying, or squeezing compositions including the attractant molecules over or onto the open cell hydrogel networks, for example, as gels, liquids, suspensions, solutions, and/or syrups over the open cell hydrogel networks. An exemplary process for applying attractant molecules and/or compositions can be seen in FIG. 1B: attractant molecules and/or compositions 8 are poured or squeezed over bait 2 which optionally contains a hook 4 and a line or lead 6.

In an alternative aspect, the attractant molecules and/or compositions can be applied to the open cell hydrogel networks by soaking the open cell hydrogel networks in aqueous solutions containing the attractant molecules and/or compositions. Further in this aspect, the attractant molecules and/or compositions can penetrate the hydrogel nodes and open cell network as they move down a concentration gradient from the more concentrated soaking solution into the open cell hydrogel networks. An exemplary process for applying attractant molecules and/or compositions can be seen in FIG. 1C: a hydrogel according to the present disclosure is suspended from line or lead 6 in a vessel holding attractant molecules and/or compositions 8, which are absorbed by the hydrogel and remain in the hydrogel upon removal of the hydrogel from the vessel.

### Attractant Molecules and Compositions

In one aspect, the attractant molecule can be inosine or another nucleoside, nucleotide, or nucleic acid derivative, including, but not limited to guanylic acid, disodium guanylate, dipotassium guanylate, calcium guanylate, inosinic acid, disodium inosinate, dipotassium inosinate, calcium inosinate, or any combination thereof. In another aspect, the attractant molecule or composition can include a scent molecule, an amino acid, a protein, a peptide, a salt, a sugar, a fish oil, a digested or partially digested aquatic organism, or any combination thereof.

In one aspect, the scent molecule can be dimethyl sulfide, toscanol, fructone, anisic aldehyde, trans-2-nonenal, melonal, trimethylamine, or any combination thereof. In another aspect, the amino acid can be L-alanine, L-glutamic acid, L-arginine, glycine, betaine, or a combination thereof. In still another aspect, the protein can be a crustacean protein, an insect protein, a fish protein, a mollusk protein, or any combination thereof. In yet another aspect, the crustacean protein can be a shrimp protein, a crab protein, or any combination thereof. In one aspect, the mollusk protein can be a clam protein, a squid protein, or any combination thereof. In one aspect, the peptide can be a hydrolyzed protein from a crustacean, an insect, a fish, a mollusk, or any combination thereof. In another aspect, the sugar can be fructose, glucose, galactose, or any combination thereof. In still another aspect, the fish oil can be menhaden oil, scad oil, sardine oil, ballyhoo oil, another bait fish oil, or a combination thereof. In one aspect, the digested or partially digested aquatic organism can be shrimp, crab, clam, squid, sardines, scad, ballyhoo, another fish, or any combination thereof.

### Further Hydrogel Components

### Thickeners

In one aspect, the disclosed open cell hydrogel networks, baits, and articles can include a polysaccharide, clay, or other thickener. In a further aspect, these polysaccharides, clays, and other thickeners can impart structural integrity to the disclosed open cell hydrogel networks, baits, and articles, can absorb and/or release ions, small molecules, and attractant molecules at the same or a different rate than the open cell hydrogel networks themselves, can act as flavors or attractants, can act as fillers, can modify the texture of the open cell hydrogel networks (e.g., imparting a more or less rubbery feel, or altering flexibility or rigidity of the open cell hydrogel networks), or any combination thereof.

In one aspect, the clay can be a crystalline phyllosilicate clay such as, for example, hectorite, smectite, montmorillonite, kaolin, or any combination thereof. In another aspect, the clay can be bentonite, a synthetic clay such as, for example, sodium magnesium silicate, or a modified clay such as, for example, quaternium-90 sepiolite or quaternium-90 montmorillonite. In one aspect, the thickener or filler can be perlite, vermiculite, mica, or a combination thereof.

In one aspect, the thickener can be a starch or a related natural product including, but not limited to, amylose, amylopectin, potato starch, wheat flour, tapioca starch, arrowroot, corn starch, katakuri starch, sago, almond flour, kudzu, rice flour, pectin, xanthan gum, guar gum, oat gum, acacia gum, gum ghatti, gum tragacanth, β-glucan from oat or barley bran, gellan gum, locust bean gum, or any combination thereof.

In still another aspect, the thickener can be a gelling agent such as, for example, agar, carrageenan, alginic acid, sodium alginate, potassium alginate, ammonium alginate, calcium alginate, konjac, or any combination thereof.

In some aspects, the disclosed open cell hydrogel networks, baits, and articles can include an agent that causes gelling of proteins already present in a composition and/or improves gel formation in combination with a gelling agent such as, for example, sodium pyrophosphate, potassium chloride, and/or sodium chloride.

In one aspect, the disclosed open cell hydrogel networks, baits, and articles can include petroleum jelly or a wax such as, for example, beeswax, carnauba wax, jojoba oil, candelilla wax, paraffin, shellac, microcrystalline wax, gum benzoic, rice bran wax, or any combination thereof.

In another aspect, the disclosed open cell hydrogel networks, baits, and articles can include a natural, synthetic, or semisynthetic polymer including, but not limited to, carboxymethylcellulose, carbomer, polyethylene glycol, pullulan, hemicellulose, Hypromellose, β-cyclodextrin, or any combination thereof.

Other thickeners and/or viscosity modifiers are also contemplated including, but not limited to, fumed silica, precipitated silica, fine talc, chalk, psyllium seed husks, casein, sodium sulfonate, calcium sulfonate, organosilicones, modified castor oil, sorbitol, mannitol, glycerol, and combinations thereof.

### Carbohydrates

In one aspect, the disclosed open cell hydrogel networks, baits, and articles can include one or more carbohydrates. In a further aspect, the carbohydrates can be monosaccharides, disaccharides, oligosaccharides, or polysaccharides. In one aspect, the carbohydrate can be selected from fructose, glucose, galactose, sucrose, lactose, maltose, trehalose, cellobiose, chitobiose, isomaltose, dextrins (including maltodextrin), dextrose, and any combination thereof. In an alternative aspect, the carbohydrate can be a syrup including a natural syrup or sweetener such as, for example, honey, maple syrup, agave nectar, molasses, brown rice syrup, coconut sugar, sucanat, date sugar, corn syrup, or a combination thereof. In any of these aspects, the carbohydrates can function as flavors or attractant molecules, can impart color, can modify permeability or structure of the open cell hydrogel networks, baits and articles, can act as filler, or any combination thereof.

### Structural Fibers and Polymers

In one aspect, the disclosed open cell hydrogel networks, baits, and articles include one or more natural fibers. In a further aspect, the natural fibers are biodegradable over time but suitable for providing structural support and integrity during use of the open cell hydrogel networks, baits, and articles. In another aspect, the natural fibers can be used unmodified or can be formed into a mesh, woven, knitted, spun, twisted, knotted, or any combination thereof.

In one aspect, the natural fiber can be selected from cellulose or a modified cellulose including, but not limited to, enzymatically hydrolyzed carboxymethyl cellulose, sodium carboxymethyl cellulose, cellulose acetate, ethyl cellulose, ethyl hydroxyethyl cellulose, hydroxypropyl cellulose, hydroxypropyl methyl cellulose, methylcellulose, ethyl methyl cellulose, microcrystalline cellulose, powdered cellulose, or any combination thereof. In another aspect, the natural fiber can be a regenerated cellulose fiber such as, for example, modal or rayon.

In another aspect, the natural fiber can be a plant-derived fiber such as, for example, flax (i.e. linen), cotton, jute, kenaf, hemp, ramie, rattan, bagasse, sisal, coconut fiber, wheat fiber, rice fiber, barley fiber, bamboo, straw, wood fiber (i.e., kraft pulp), vegetable fiber, or any combination thereof.

In an alternative aspect, the natural fiber can be an animal-derived fiber such as, for example, collagen, keratin, fibroin, spidroin, sinew, wool or felted wool, catgut, angora, mohair, alpaca, cashmere, camel hair, horse hair, fiber from feathers, chitin, chitosan, tendon fibers, other hair, or any combination thereof.

### Animal-Derived Products

In one aspect, the disclosed open cell hydrogel networks, baits, and articles can include one or more animal-derived products not already mentioned. In a further aspect, the animal-derived products can be selected from collagen, hydrolyzed collagen, and/or gelatin. In a further aspect, the collagen, hydrolyzed collagen, and/or gelatin can come from any common industrial source including, but not limited to, cattle, fish, horses, pigs, rabbits, and/or chickens. In another aspect, the animal-derived products can include keratin, hyaluronic acid, or tissues such as, for example, tendon, ligament, muscle, organ tissue, cartilage, hide or skin, scales, feathers, hair, horn, bone, egg whites, egg yolks, egg proteins such as, for example, albumin, and/or combinations thereof. In any of these aspects, the animal-derived product can provide structure to the disclosed open cell hydrogel networks, baits, and articles, or can serve as a flavor or attractant molecule.

### Mineral-Derived Components and Inorganic Salts

In one aspect, the disclosed open cell hydrogel networks, baits, and articles can include one or more salts and/or mineral derived compounds. In a further aspect, the mineral derived compound can include quicklime (calcium oxide), sodium sulfite, sodium bisulfite, sodium metabisulfite, potassium sulfite, calcium sulfite, calcium hydrogen sulfite, potassium hydrogen sulfite, potassium nitrite, sodium nitrite, sodium nitrate, potassium nitrate, sodium phosphate, potassium phosphate, calcium phosphate, ammonium phosphate, magnesium phosphate, magnesium oxide, calcium silicate, sodium silicate, silicon dioxide, magnesium silicate, talc, sodium aluminosilicate, potassium aluminum silicate, calcium aluminosilicate, zinc silicate, aluminum silicate, potassium silicate, or any combination thereof.

In another aspect, the salt can be potassium chloride, calcium chloride, ammonium chloride, magnesium chloride, stannous chloride, sodium sulfate, sodium chloride, sodium bisulfate, potassium sulfate, potassium bisulfate, calcium sulfate, ammonium sulfate, magnesium sulfate, copper(II) sulfate, aluminum sulfate, aluminum sodium sulfate, aluminum potassium sulfate, aluminum ammonium sulfate, or any combination thereof.

In any of these aspects, the salt and/or mineral derived compound may serve a variety of purposes including, but not limited to, acting as a filler, acting as a buffer or otherwise adjusting the pH of the microenvironment of the disclosed open cell hydrogel networks, baits, and articles, assisting in biodegradation, altering the opacity of the disclosed open cell hydrogel networks, baits, and articles, serving as a flavor or attractant molecule, imparting a color to the disclosed open cell hydrogel networks, baits, and articles, or any combination thereof.

### Organic Acids, Salts of Organic Acids, and Inorganic Bases

In one aspect, the disclosed open cell hydrogel networks, baits, and articles can include one or more organic acids or fatty acids including, but not limited to, sorbic acid, benzoic acid, glutamic acid, formic acid, acetic acid, propionic acid, ascorbic acid, lactic acid, citric acid, malic acid, tartaric acid, adipic acid, fumaric acid, stearic acid, gluconic acid, or any combination thereof.

In another aspect, the disclosed open cell hydrogel networks, baits, and articles can include a salt of an organic acid or fatty acid including, but not limited to, sodium sorbate, potassium sorbate, calcium sorbate, sodium benzoate, potassium benzoate, calcium benzoate, sodium formate, calcium formate, potassium acetate, sodium acetate, sodium diacetate, calcium acetate, ammonium acetate, sodium dehydroacetate, sodium propionate, calcium propionate, potassium propionate, sodium ascorbate, calcium ascorbate, potassium ascorbate, sodium lactate, potassium lactate, calcium citrate, magnesium citrate, sodium malate, potassium malate, calcium malate, calcium tartrate, sodium adipate, potassium adipate, ammonium adipate, monosodium fumarate, potassium fumarate, calcium fumarate, ammonium fumarate, ammonium ferric citrate, calcium disodium EDTA, disodium EDTA, sodium tartrate, potassium tartrate, magnesium stearate, calcium stearate, sodium gluconate, potassium gluconate, calcium gluconate, ferrous gluconate, magnesium gluconate, ferrous lactate, monosodium glutamate, monopotassium glutamate, calcium diglutamate, monoammonium glutamate, magnesium glutamate, zinc acetate, or any combination thereof.

In another aspect, the disclosed open cell hydrogel networks, baits, and articles can include one or more inorganic bases such as, for example, sodium hydroxide, potassium hydroxide, calcium hydroxide, ammonium hydroxide or ammonia, magnesium hydroxide, or any combination thereof.

In yet another aspect, the disclosed open cell hydrogel networks, baits, and articles can include one or more effervescent or non-effervescent carbonate such as, for example, sodium carbonate, sodium bicarbonate, sodium sesquicarbonate, potassium carbonate, potassium bicarbonate, ammonium carbonate, ammonium bicarbonate, magnesium carbonate, magnesium bicarbonate, ferrous carbonate, calcium carbonate, or any combination thereof.

In a further aspect, any of these compounds may perform a function including, but not limited to, acting as a filler, acting as a buffer or pH adjustment agent, providing flavor or acting as an attractant molecule, providing color, providing a source of carbon, nitrogen, oxygen, or phosphorus as a nutrient source, chelating heavy metals, acting as a preservative, modifying the texture of the open cell hydrogel networks, baits, and articles, providing effervescence to the aquatic environment surrounding the bait, or any combination thereof.

### Additives for Modifying Color, Reflectance, and Clarity

In one aspect, the disclosed open cell hydrogel networks, baits, and articles can include one or more components intended to alter the color, reflectance, clarity, opacity, or other aspects of the appearance of the open cell hydrogel networks, baits, and articles.

In a further aspect, the color-modifying component can be an azo dye or pigment including a pyrazolone dye, a xanthene dye, an indigoid dye, a triarylmethane dye, a phthalocyanine dye, or any combination thereof. In a further aspect, the pyrazolo dye can be selected from tartrazine, orange B, mordant red 19, yellow 2G, acid yellow 17, pigment yellow 13, pigment red 38, or any combination thereof. In a further aspect, the xanthene day can be selected from fluorescein, eosin B, eosin Y, rhodamine B, rhodamine 123, or any combination thereof. In still another aspect, the triarylmethane dye can be selected from methyl violet 2B, methyl violet 6B, methyl violet 10B, pararosaniline, fuchsine, new fuchsine, fuchsine acid, phenolphthalein, phenol red, chlorophenol red, cresol red, bromocresol purple, bromocresol green, malachite green, brilliant green, brilliant blue FCF, Victoria blue B, Victoria blue FBR, Victoria blue BO, Victoria blue FGA, Victoria blue 4R, Victoria blue R, or any combination thereof. In one aspect, the color-modifying component can be a carotenoid such as, for example, α-carotene, β-carotene, γ-carotene, lycopene, β-apo-8′-carotenal, the ethyl ester of β-apo-8′-carotenic acid, or any combination thereof. In another aspect, the carotenoid can be a xanthophyll such as, for example, flavoxanthin, lutein, cryptoxanthin, rubixanthin, violaxanthin, rhodaxanthin, canthaxanthin, zeaxanthin, citranaxanthin, astaxanthin, or any combination thereof. In still another aspect, the color-modifying component can be an anthocyanin.

In still another aspect, the color-modifying component can be an approved food dye such as, for example, curcumin, riboflavin, riboflavin-5′-phosphate, tartrazine, quinoline yellow WS, riboflavin-5′-sodium phosphate, sunset yellow FCF, cochineal, carmoisine, erythrosine, allura red AC, indigo carmine, brilliant blue FCF, chlorophylls and chlorophyllins, copper complexes of chlorophylls and chlorophyllins, fast green FCF, caramel, carbon black, vegetable carbon, annatto, paprika oleoresin, saffron, or any combination thereof.

In one aspect, the color-modifying component can be a natural dye such as, for example, those known as or sourced from cochineal, Indian yellow (i.e., cow urine), lac insect, murex snail, octopus or cuttlefish, catechu, gamboge tree resin, chestnut hulls, Himalayan rhubarb root, indigofera leaves, kamala seed pods, madder root, mangosteen peel, pomegranate rind, teak leaf, weld herb, black walnut hulls, flavonoids, betalains, and combinations thereof.

In some aspects, the color-modifying component can be a mineral or mineral derived pigment including, but not limited to, calcium carbonate, chalk, titanium dioxide, iron oxides, iron hydroxides, and combinations thereof.

In other aspects, the color-modifying component can impart shine or iridescence to the disclosed open cell hydrogel networks, baits, and articles. In some aspects, shine and/or iridescence can mimic the appearance of prey organisms (e.g., insects, scales of smaller fish) to entice fish. Further in this aspect, the color-modifying component can be a metal-coated or oxide-coated mica or other interference pigment that imparts a structural color to the open cell hydrogel networks, or can be a glitter. In a further aspect, the glitter can include plastic, real or synthetic mica, minerals, insect parts (e.g., beetle wings), glass, aluminum, fish scales, or any combination thereof.

### Materials for Adjusting Buoyancy

In one aspect, the disclosed open cell hydrogel networks, baits, and articles can include one or more materials or components useful for adjusting buoyancy. In a further aspect, the materials or components useful for adjusting buoyancy can cause the open cell hydrogel networks, baits, and articles to sink, to float, or to maintain a specific position below the water level. In some aspects, buoyancy may be dependent upon temperature or salinity of the water.

In one aspect, the disclosed open cell hydrogel networks, baits, and articles incorporate a natural lightweight material such as, for example, wood or cork, that can help the open cell hydrogel networks, baits, and articles float near the water surface.

In another aspect, the disclosed open cell hydrogel networks, baits, and articles can include a wire or a heavy fishing hook, metal shavings, metal cores, metal pellets or metal balls (such as, for example, ball bearings), or clay in order to add weight to the disclosed materials and assist in sinking of the open cell hydrogel networks, baits, and articles. In one aspect, the added metallic components can be selected from lead, lead-free pewter, steel, tungsten, brass, bismuth, or a combination thereof. In an alternative aspect, a plastic or synthetic material such as, for example, high density composite resin or an epoxy glue or resin can be used to modify weight in a similar manner. In yet another aspect, a foam material such as, for example, polyurethane or polystyrene foam can be incorporated into the open cell hydrogel networks, baits, and articles to increase buoyancy.

In still another aspect, the disclosed open cell hydrogel networks, baits, and articles can include a non-permeable internal cavity (for example, lined with plastic or glass) enclosing a material that is denser or less dense than the open cell hydrogel networks, baits, and articles. In another aspect, the material can be an entrapped gas or air or can be fresh water, salt water, or another fluid. In some aspects, the open cell hydrogel networks, baits, and articles are configured such that fluid can be added or removed to adjust the buoyancy of the disclosed open cell hydrogel networks, baits, and articles.

### Materials for Hastening Biodegradation

In one aspect, the disclosed open cell hydrogel networks, baits, and articles disclosed herein can optionally include one or more components that favors or enhances biodegradation of the open cell hydrogel networks, baits, and articles. In some aspects, the disclosed open cell hydrogel networks, baits, and articles can be seeded with bacteria and/or fungi known to degrade the components of the bait. In one aspect, the bacteria and/or fungi can consist of mixed strains of organisms (i.e., more than one species or more than one strain) for more effective biodegradation. In one aspect, the microorganisms can be genetically engineered or can incorporate plasmids expressing genes useful for hydrogel degradation. In some aspects, natural fibers and/or porous materials can be incorporated in the open cell hydrogel networks in order to provide a favorable environment for bacterial and/or fungal growth.

In another aspect, trace nutrients useful for microbial metabolism can be added to the disclosed open cell hydrogel networks, baits, and articles, including, but not limited to, trace minerals, electron acceptors, electron donors, carbon sources, nitrogen sources, oxygen sources, phosphorus sources, and the like. In one aspect, the open cell hydrogel networks, baits, and articles can include one or more agents useful for adjusting the pH of the local environment of the bait in order to facilitate microbial growth.

In an alternative aspect, one or more components of the hydrogel can be selected from biodegradable plastics such as, for example, polyhydroxyalkanoates, polylactic acids, starch blends including gelatinized starches, cellulose-based plastics, lignin-based plastics, polyglycolic acid, polybutylene succinate, polycaprolactone, polyvinyl alcohol, or any combination thereof.

Release of Components from Open Cell Hydrogel Networks

In any of the above aspects, attractant molecules and/or compositions are taken up into the open cell hydrogel networks after applying the attractant molecules and/or compositions by any of the disclosed means. In one aspect, absorption of the attractant molecules and/or compositions into the open cell hydrogel networks provides more control over release of the attractant molecules and/or compositions than simply coating an existing bait with the attractant molecules and/or compositions.

In another aspect, by fine-tuning the properties of the open cell hydrogel networks, the rate of release of the attractant molecules and/or compositions can be controlled. In a further aspect, the attractant molecules and/or compositions begin to release from the open cell hydrogel networks upon placement of the open cell hydrogel networks in an aqueous medium such as, for example, water in a lake, pond, stream, creek, or river. A schematic of attractant molecules and/or compositions releasing from the hydrogel is shown in FIG. 1D. In one aspect, the network of hydrogel including the nodes, aggregated nodes, and fibrils provides a slow release of the attractant molecules and/or compositions, while the open cell network existing between the nodes and fibrils provides for a rapid release of the attractant molecules and/or compositions. In a further aspect, permeability and release of active compounds from the open cell hydrogel networks are tunable based on polymer concentration and spacing, polymer identity, pore size in the open hydrogel network, and related factors.

In one aspect, release of the compounds can last for from about 1 minute to about 10 minutes, or can take about 1, 2, 3, 4, 5, 6, 7, 8, 9, or about 10 minutes, or a combination of any of the foregoing values, or a range encompassing any of the foregoing values.

Also disclosed herein are methods for attracting aquatic organisms, the methods including placing the open cell hydrogel networks and/or baits disclosed herein into a body of water containing the aquatic organisms. In one aspect, the body of water can be freshwater, saltwater, or brackish water. In another aspect, the body of water can have a pH of from about 5.6 to about 8.5, or of about 5.6, 6, 6.5, 7, 7.5, 8, or about 8.6, or a combination of any of the foregoing values, or a range encompassing any of the foregoing values.

### Further Applications of the Open Cell Hydrogel Networks

### Filters

In some aspects, the open cell hydrogel networks disclosed herein can be used as filters.

In a further aspect, the open cell hydrogel networks can be used to filter objects having a characteristic size on the order of micrometers such as, for example, debris, cells, and biological materials in suspension, and/or microorganisms including but not limited to bacteria from liquid cell culture media.

### Drug Delivery Devices

In one aspect, disclosed herein are drug delivery devices incorporating the open cell hydrogel networks disclosed herein. In another aspect, the drug delivery device can be an implant, a transdermal patch, a punctal plug, a contact lens, nanoparticles, a suppository, a wound dressing, or any combination thereof. In any of these aspects, the drug delivery device can be biocompatible.

In another aspect, the porous network structure of the disclosed open cell hydrogel networks can hold up to 50% or more (v/v) of a liquid phase. Without wishing to be bound by theory, since spacing in the gel is small, convection is suppressed and diffusion is the sole mechanism for delivering a drug. In a further aspect, the open cell hydrogel networks can be geometrically tailored to release a drug in order to achieve a prescribed concentration profile of the drug in the surrounding tissue. In still another aspect, without wishing to be bound by theory, since the open cell hydrogel networks have a low modulus of elasticity, the size of the drug delivery devices can be increased.

In still another aspect, if designed using collagen or another biological polymer, the open cell hydrogel networks can be engineered to break down in the body at a controlled rate (e.g., at a rate that is slower than the rate of drug release). Further in this aspect, the need for surgical removal of an implanted device as disclosed could be avoided in the case of biodegradable polymers and open cell hydrogel networks.

In one aspect, the drug delivery devices disclosed herein contain at least one pharmaceutical agent. In another aspect, the drug delivery devices release the at least one pharmaceutical agent over a period of from about 1 week to about 6 months, or over a period of about 1, 2, 3, or 4 weeks, or about 1, 2, 3, 4, 5, or about 6 months, or a combination of any of the foregoing values, or a range encompassing any of the foregoing values. In some aspects, the pharmaceutical agent can be an anti-cancer agent.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. An article comprising a polymeric material wherein the article is suited to extended periods of immersion in an aqueous environment and wherein the article is permeable to small molecules, wherein small molecules contained within the article can be released from the article into the aqueous environment

Aspect 2. The article of Aspect 1, wherein the polymeric material comprises an open cell hydrogel network comprising a plurality of hydrogel nodes attached to one another, wherein at least a portion of the nodes cluster to form a plurality of aggregated hydrogel nodes.

Aspect 3. The article of Aspect 1, wherein the polymeric material comprises an open cell hydrogel network comprising a plurality of hydrogel nodes crosslinked with one another.

Aspect 4. The article any one of Aspects 1-3, wherein the hydrogel nodes comprise an average polymer weight percent of from 10 wt % to 80 wt % polymer.

Aspect 5. The article of any one of Aspects 2-4, wherein the hydrogel nodes have an average diameter of from about 10 nm to about 500 μm.

Aspect 6. The article of any one of Aspects 1-5, wherein the open cell network has a characteristic dimension of from about 10 nm to about 1 mm.

Aspect 7. The article of any one of Aspects 1-5, wherein the open cell network has a characteristic dimension of from about 100 nm to about 500 μm.

Aspect 8. The article of any one of Aspects 1-5, wherein the open cell network has a characteristic dimension of from about 1 μm to about 100 μm.

Aspect 9. The article of any one of Aspects 1-8, wherein at least a portion of the nodes are attached to one another through direct contact or through fibrils.

Aspect 10. The article of any one of Aspects 1-9, wherein the polymeric material has a permeability of from about 1×10⁻¹⁵ m² to about 1×10⁻⁴ m².

Aspect 11. The article of any one of Aspects 1-9, wherein the polymeric material has a permeability of from about 5×10⁻¹² m² to about 1×10⁻⁸ m².

Aspect 12. The article of any one of Aspects 1-11, wherein the polymeric material comprises from about 20% to about 95% water by mass.

Aspect 13. The article of any one of Aspects 1-12, wherein the polymeric material comprises about 10% polymer by mass to about 30% polymer by mass.

Aspect 14. The article of any one of Aspects 1-13, wherein the polymeric material comprises a modulus of from about 10 kPa to about 100 MPa.

Aspect 15. The article of any one of Aspects 1-13, wherein the polymeric material comprises a modulus of from about 10 kPa to about 1 MPa.

Aspect 16. The article of any one of Aspects 1-15, wherein the hydrogel nodes comprise the polymerization product of one or more monomers comprising hydroxyethylmethacrylate (HEMA), a polyethylene glycol (PEG) monomer, a polypropylene oxide (PPO) monomer, lactic acid, glycolic acid, N-isopropylacrylamide (NIPAM), caprolactone, R-3-hydroxybutyrate, vinyl acetate, ethylene, acrylamide, methacrylamide, dimethacrylamide, acrylate, methacrylate, acrylic acid, methacrylic acid, C₂-C₈ alkyl acrylic acids, vinyl ester, vinyl amide, vinyl amine, poly(ethylene glycol) diacrylate (PEGDA), N-vinylpyrrolidone, hydroxyethyl acrylate, hydroxypropyl acrylate, trimethylammonium 2-hydroxypropylmethacrylate, dimethylaminoethyl methacrylate (DMAEMA), dimethylaminoethylmethacrylamide, allyl alcohol, vinylpyridine, glycerol methacrylate, N-(1,1-dimethyl-3-oxobutyl)acrylamide, or any combination thereof.

Aspect 17. The article of any one of Aspects 1-16, wherein the polymeric material comprises an open cell hydrogel network comprising a plurality of hydrogel nodes attached to one another, wherein the hydrogel nodes comprise the polymerization product of hydroxyethylmethacrylate (HEMA), wherein the polymeric material comprises about 10% by mass to about 30% by mass HEMA, and wherein the polymeric material has a permeability of from about 5×10¹² m² to about 1×10⁻⁸ m².

Aspect 18. The article of Aspect 17, wherein the polymeric material comprises an open cell hydrogel network comprising a plurality of hydrogel nodes crosslinked with one another, wherein the hydrogel is the polymerization product of hydroxyethylmethacrylate (HEMA) and N,N′-methylenebisacrylamide (BIS).

Aspect 19. A polymeric material produced by the method comprising:
a. admixing a crosslinker, an initiator, and a monomer in water to form a first solution; and
b. admixing the first solution with a catalyst to polymerize the monomer to form the hydrogel;
wherein water is present in an amount greater than the solvation limit of the monomer.

Aspect 20. The polymeric material of Aspect 19, wherein the monomer comprises hydroxyethylmethacrylate (HEMA), a polyethylene glycol (PEG) monomer, a polypropylene oxide (PPO) monomer, lactic acid, glycolic acid, N-isopropylacrylamide (NIPAM), caprolactone, R-3-hydroxybutyrate, vinyl acetate, ethylene, acrylamide, methacrylamide, dimethacrylamide, acrylate, methacrylate, acrylic acid, methacrylic acid, C₂-C₈ alkyl acrylic acids, vinyl ester, vinyl amide, vinyl amine, poly(ethylene glycol) diacrylate (PEGDA), N-vinylpyrrolidone, hydroxyethyl acrylate, hydroxypropyl acrylate, trimethylammonium 2-hydroxypropylmethacrylate, dimethylaminoethyl methacrylate (DMAEMA), dimethylaminoethylmethacrylamide, allyl alcohol, vinylpyridine, glycerol methacrylate, N-(1,1-dimethyl-3-oxobutyl)acrylamide, or any combination thereof.

Aspect 21. The polymeric material of Aspect 20, wherein the method further comprises removing at least one functional group from the hydrogel by base-catalyzed hydrolysis.

Aspect 22. The polymeric material of Aspect 21, wherein the at least one functional group comprises an acetate group.

Aspect 23. The polymeric material of any one of Aspects 19-22, wherein the crosslinker comprises two or more acryloyl groups, methacryloyl groups, or a combination thereof.

Aspect 24. The polymeric material of any one of Aspects 19-22, wherein the crosslinker comprises N,N′-methylenebisacrylamide (BIS), ethylene glycol dimethacrylate (EGDMA), or a combination thereof.

Aspect 25. The polymeric material of any one of Aspects 19-24, wherein the initiator comprises thermal initiator, a photoinitiator, a chemical initiator, or any combination thereof.

Aspect 26. The polymeric material of any one of Aspects 19-24, wherein the initiator comprises ammonium persulfate, potassium persulfate, sodium metabisulfite, azoisobutyronotrile (AIBN), potassiumperoxodisulfate, dibenzoyl peroxide, hydrogen peroxide, sodium percarbonate, benzoin methyl ether, 1-hydroxycyclohexylphenylketone, or any combination thereof.

Aspect 27. The polymeric material of any one of Aspects 19-26, wherein the polymer comprises HEMA, the crosslinker comprises BIS, the initiator comprises ammonium persulfate, and the catalyst comprises TEMED.

Aspect 28. The polymeric material of any one of Aspects 19-27, wherein the weight ratio of monomer to water is from about 10:90 to about 80:20.

Aspect 29. The polymeric material of any one of Aspects 19-28, wherein the polymeric material comprises aggregated nodes having an average diameter of from about 10 nm to about 500 μm.

Aspect 30. The polymeric material of any one of Aspects 19-29, wherein the polymeric material comprises an open cell network having a characteristic dimension of from about 10 nm to about 1 mm.

Aspect 31. The polymeric material of any one of Aspects 19-29, wherein the polymeric material comprises an open cell network having a characteristic dimension of from about 100 nm to about 500 μm.

Aspect 32. The polymeric material of any one of Aspects 19-29, wherein the polymeric material comprises an open cell network having a characteristic dimension of from about 1 μm to about 100 μm.

Aspect 33. The polymeric material of any one of Aspects 19-32, wherein the polymeric material has a permeability of from about 1×10⁻¹⁵ m² to about 1×10⁻⁴ m².

Aspect 34. The polymeric material of any one of Aspects 19-32, wherein the polymeric material has a permeability of from about 5×10⁻¹² m² to about 1×10⁻⁸ m².

Aspect 35. The polymeric material of any one of Aspects 19-34, wherein the polymeric material comprises from about 20% to about 95% water by mass.

Aspect 36. The polymeric material of any one of Aspects 19-35, wherein the polymeric material comprises about 10% polymer by mass to about 30% polymer by mass.

Aspect 37. The polymeric material of any one of Aspects 19-36, wherein the polymeric material comprises an open cell hydrogel network comprising a plurality of hydrogel nodes attached to one another, wherein the hydrogel nodes comprise the polymerization product of hydroxyethylmethacrylate (HEMA), wherein the polymeric material comprises about 10% by mass to about 30% by mass HEMA, and wherein the polymeric material has a permeability of from about 5×10⁻¹² m² to about 1×10⁻⁸ m².

Aspect 38. The article of any one of Aspects 2-18 or the polymeric material of any one of Aspects 19-37, wherein the polymeric material comprises a modulus of from about 10 kPa to about 100 MPa.

Aspect 39. The article of any one of Aspects 2-18 or the polymeric material of any one of Aspects 19-37, wherein the polymeric material comprises a modulus of from about 10 kPa to about 1 MPa.

Aspect 40. The article of any one of Aspects 2-18 or the polymeric material of any one of Aspects 19-39, wherein the polymeric material is charged.

Aspect 41. The article of any one of Aspects 2-18 or the polymeric material of any one of Aspects 19-39, wherein the polymeric material is neutral.

Aspect 42. The article of any one of Aspects 2-18 or the polymeric material of any one of Aspects 19-41, wherein the polymeric material is biodegradable.

Aspect 43. The article of any one of Aspects 2-18 or the polymeric material of any one of Aspects 19-41, wherein the polymeric material is not biodegradable.

Aspect 44. The article of any one of Aspects 2-18 or the polymeric material of any one of Aspects 19-43, wherein the polymeric material comprises at least one color-modifying agent.

Aspect 45. The article or polymeric material of Aspect 44, wherein the color-modifying agent comprises a solid pigment, a vinyl sulfone dye, an azo dye or pigment, a pyrazolone dye, a xanthene dye, an indigoid dye, a triarylmethane dye, a phthalocyanine dye, a food dye, a mineral-derived pigment, a coated mica, glitter, or a combination thereof, and wherein the pigment is admixed with the crosslinker, the initiator, and the monomer in water prior to polymerization.

Aspect 46. The article or polymeric material of Aspect 44 or 45, wherein the pigment comprises a chromophore chemically bound to the polymeric material or to a monomer used to form the polymeric material, entrapped within but not chemically bound to the polymeric material, or a combination thereof.

Aspect 47. The article or polymeric material of any one of Aspects 2-46, wherein the polymeric material further comprises a filler, a thickener, or any combination thereof.

Aspect 48. The article or polymeric material of Aspect 47, wherein the filler comprises a mesh, a matrix, or a plurality of fibers.

Aspect 49. The article or polymeric material of Aspect 47 or 48, wherein the filler comprises a natural fiber, a natural or synthetic clay, a carbohydrate or modified carbohydrate, a gelling agent, a gum, a wax, polyethylene, polypropylene, nylon, melamine foam, a metal, or any combination thereof.

Aspect 50. The article or polymeric material of any one of Aspects 47-49, wherein the polymeric material forms an outer layer around the filler or thickener.

Aspect 51. The article or polymeric material of any one of Aspects 47-50, wherein the filler or thickener is distributed throughout the polymeric material.

Aspect 52. The article or polymeric material of any one of Aspects 2-51, further comprising a preservative, an antibacterial agent, an antimicrobial agent, an antioxidant, or any combination thereof.

Aspect 53. The article or polymeric material of any one of Aspects 2-52, further comprising a buoyancy modifier.

Aspect 54. The article or polymeric material of Aspect 53, wherein the buoyancy modifier comprises a polymeric foam, a fluid, a resin, a metal, wood, cork, clay, or any combination thereof.

Aspect 55. The article or polymeric material of Aspect 54, wherein the metal comprises a wire, a hook, metal shavings, metal pellets, metal balls, a solid metal core, or any combination thereof.

Aspect 56. The article or polymeric material of Aspect 54 or 55, wherein the metal comprises lead, pewter, steel, tungsten, bismuth, brass, or any combination thereof.

Aspect 57. The article or polymeric material of any one of Aspects 2-56, further comprising a component that enhances biodegradability.

Aspect 58. The article or polymeric material of Aspect 57, wherein the component that enhances biodegradability comprises bacteria, fungi, a biodegradable plastic, a trace mineral, a source of carbon, nitrogen, oxygen, or phosphorus, or a combination thereof.

Aspect 59. The article or polymeric material of any one of Aspects 2-58, further comprising a mineral-derived component, a salt, an organic acid or salt thereof, an inorganic base, a carbonate, or any combination thereof.

Aspect 60. The article or polymeric material of any one of Aspects 2-59 wherein the mineral-derived component comprises quicklime (calcium oxide), sodium sulfite, sodium bisulfite, sodium metabisulfite, potassium sulfite, calcium sulfite, calcium hydrogen sulfite, potassium hydrogen sulfite, potassium nitrite, sodium nitrite, sodium nitrate, potassium nitrate, sodium phosphate, potassium phosphate, calcium phosphate, ammonium phosphate, magnesium phosphate, magnesium oxide, calcium silicate, sodium silicate, silicon dioxide, magnesium silicate, talc, sodium aluminosilicate, potassium aluminum silicate, calcium aluminosilicate, zinc silicate, aluminum silicate, potassium silicate, or any combination thereof.

Aspect 61. The article or polymeric material of any one of Aspects 2-60, wherein the salt comprises potassium chloride, calcium chloride, ammonium chloride, magnesium chloride, stannous chloride, sodium sulfate, sodium chloride, sodium bisulfate, potassium sulfate, potassium bisulfate, calcium sulfate, ammonium sulfate, magnesium sulfate, copper(II) sulfate, aluminum sulfate, aluminum sodium sulfate, aluminum potassium sulfate, aluminum ammonium sulfate, or any combination thereof.

Aspect 62. The article or polymeric material of any one of Aspects 2-61, wherein the organic acid or salt thereof comprises sodium sorbate, potassium sorbate, calcium sorbate, sodium benzoate, potassium benzoate, calcium benzoate, sodium formate, calcium formate, potassium acetate, sodium acetate, sodium diacetate, calcium acetate, ammonium acetate, sodium dehydroacetate, sodium propionate, calcium propionate, potassium propionate, sodium ascorbate, calcium ascorbate, potassium ascorbate, sodium lactate, potassium lactate, calcium citrate, magnesium citrate, sodium malate, potassium malate, calcium malate, calcium tartrate, sodium adipate, potassium adipate, ammonium adipate, monosodium fumarate, potassium fumarate, calcium fumarate, ammonium fumarate, ammonium ferric citrate, calcium disodium EDTA, disodium EDTA, sodium tartrate, potassium tartrate, magnesium stearate, calcium stearate, sodium gluconate, potassium gluconate, calcium gluconate, ferrous gluconate, magnesium gluconate, ferrous lactate, monosodium glutamate, monopotassium glutamate, calcium diglutamate, monoammonium glutamate, magnesium glutamate, zinc acetate, sorbic acid, benzoic acid, glutamic acid, formic acid, acetic acid, propionic acid, ascorbic acid, lactic acid, citric acid, malic acid, tartaric acid, adipic acid, fumaric acid, stearic acid, gluconic acid, or any combination thereof.

Aspect 63. The article or polymeric material of any one of Aspects 2-62, wherein the inorganic base comprises sodium hydroxide, potassium hydroxide, calcium hydroxide, ammonium hydroxide or ammonia, magnesium hydroxide, or any combination thereof.

Aspect 64. The article or polymeric material of any one of Aspects 2-63, wherein the carbonate comprises sodium carbonate, sodium bicarbonate, sodium sesquicarbonate, potassium carbonate, potassium bicarbonate, ammonium carbonate, ammonium bicarbonate, magnesium carbonate, magnesium bicarbonate, ferrous carbonate, calcium carbonate, or any combination thereof.

Aspect 65. A bait formed from the article of any one of Aspects 1-18 or 38-64 or the polymeric material of any one of Aspects 19-64.

Aspect 66. The bait of Aspect 65, wherein the bait is formed in the shape of an aquatic animal, an insect, a worm, or fish eggs.

Aspect 67. The bait of Aspect 65 or 66, further comprising at least one attractant molecule or composition.

Aspect 68. The bait of Aspect 67, wherein the attractant molecule comprises inosine, guanylic acid or a salt thereof, inosinic acid or a salt thereof, or any combination thereof.

Aspect 69. The bait of Aspect 67, wherein the attractant molecule of composition comprises a scent molecule, an amino acid, a protein, a peptide, a salt, a carbohydrate, a fish oil, a digested or partially digested aquatic organism, or a combination thereof.

Aspect 70. The bait of Aspect 68, wherein the scent molecule comprises dimethyl sulfide, toscanol, fructone, anisic aldehyde, trans-2-nonenal, melonal, trimethylamine, or any combination thereof.

Aspect 71. The bait of Aspect 68, wherein the amino acid comprises L-alanine, L-glutamic acid, L-arginine, glycine, betaine, or a combination thereof.

Aspect 72. The bait of Aspect 68, wherein the protein comprises a crustacean protein, an insect protein, a fish protein, a mollusk protein, or any combination thereof.

Aspect 73. The bait of Aspect 72, wherein the crustacean protein comprises a shrimp protein, a crab protein, or any combination thereof.

Aspect 74. The bait of Aspect 72, wherein the mollusk protein comprises a clam protein, a squid protein, or any combination thereof.

Aspect 75. The bait of Aspect 69, wherein the peptide comprises a hydrolyzed protein from a crustacean, an insect, a fish, a mollusk, or any combination thereof.

Aspect 76. The bait of Aspect 69, wherein the sugar comprises fructose, glucose, galactose, or any combination thereof.

Aspect 77. The bait of Aspect 69, wherein the fish oil comprises menhaden oil, scad oil, sardine oil, ballyhoo oil, another bait fish oil, or a combination thereof.

Aspect 78. The bait of Aspect 69, wherein the digested or partially digested aquatic organism comprises shrimp, crab, clam, squid, sardines, scad, ballyhoo, another fish, or any combination thereof.

Aspect 79. The bait of any one of Aspects 67-78, wherein the attractant molecule or composition is released from the bait over a period of time when the bait is placed in water.

Aspect 80. The bait of Aspect 79, wherein the period of time is from about 1 minute to about 10 minutes.

Aspect 81. A method for attracting aquatic organisms, the method comprising placing the bait of any one of Aspects 67-80 into a body of water containing the aquatic organisms.

Aspect 82. The method of Aspect 81, wherein the body of water comprises freshwater, saltwater, or brackish water.

Aspect 83. The method of Aspect 81 or 82, wherein the body of water comprises a pH of from about 5.6 to about 8.5.

Aspect 84. A filter formed from the article of any one of Aspects 1-18 or 38-64 or the polymeric material of any one of Aspects 19-64.

Aspect 85. A drug delivery device incorporating the article of any one of Aspects 1-16 or 36-62 or the polymeric material of any one of Aspects 19-64.

Aspect 86. The drug delivery device of Aspect 85, wherein the drug delivery device comprises an implant, a transdermal patch, a punctal plug, a contact lens, nanoparticles, a suppository, a wound dressing, or any combination thereof.

Aspect 87. The drug delivery device of Aspect 85 or 86, wherein the drug delivery device is biocompatible.

Aspect 88. The drug delivery device of any one of Aspects 85-87, further comprising at least one pharmaceutical agent.

Aspect 89. The drug delivery device of Aspect 88, wherein the drug delivery device releases the at least one pharmaceutical agent over a period of from about 1 week to about 6 months.

Aspect 90. The drug delivery device of Aspect 88 or 89, wherein the at least one pharmaceutical agent comprises an anti-cancer agent.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Gel Preparation

A 150 mL stock solution of 2% (w/v) N,N′-methylenebisacrylamide (BIS) was prepared by dissolving 3 g of BIS in 150 mL of water. A 15 mL stock solution of 10% (w/v) ammonium persulfate (APS) was prepared by dissolving 1.5 g APS in 15 mL of water. A 15 mL stock solution of 10% (w/v) tetramethylethylenediamine (TEMED) was prepared by dissolving 1.5 g of TEMED in 15 mL of water.

1 L of molding solution was prepared using the proportions in Table 1:

TABLE 1
Preparation of Molding Solution

| Component | Form | Volume (mL) |
|---|---|---|
| BIS | 2% (w/v) in water | 150 |
| 10% APS | 10% (w/v) in water | 15 |
| hydroxyethylmethacrylate (HEMA) | liquid | 200 |
| Water | N/A | 620 |

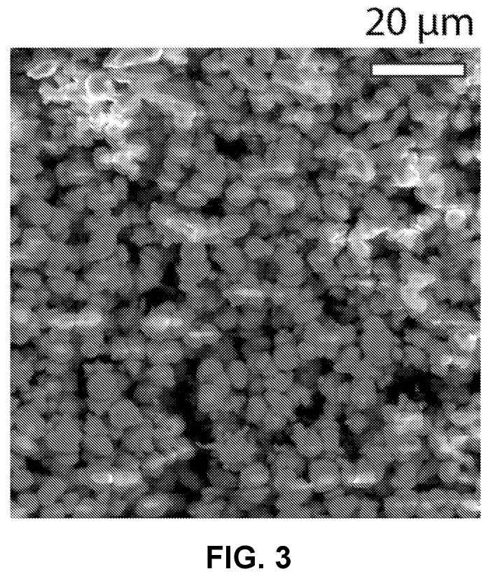

150 mL of 10% (w/v) TEMED solution as prepared above was added to 850 mL of molding solution to polymerize the molding solution. The gel cured quickly; cure time was adjustable by changing the concentrations of TEMED and APS solutions. A SEM image of an exemplary gel is presented in FIG. 3.

### Example 2: Permeability of the Open Cell Hydrogel Networks

Open cell hydrogel networks were prepared according to the process disclosed herein with different volume percentages of HEMA. Permeability (k) for these gels is presented in Table 2:

TABLE 2
Permeability of Gels with Different Amounts of HEMA

| % HEMA (v/v) | k (m²) |
|---|---|
| 15 | 5.45 × 10⁻¹² |
| 17.5 | 3.84 × 10⁻¹² |
| 20 | 3.32 × 10⁻¹² |
| 22.5 | 2.75 × 10⁻¹² |

It was observed that as volume percentage of HEMA increases, permeability of the open cell hydrogel networks decreases.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. (canceled)

2. An article comprising a polymeric material, wherein the polymeric material comprises an open cell hydrogel network comprising a plurality of hydrogel nodes attached to one another, wherein at least a portion of the nodes cluster to form a plurality of aggregated hydrogel nodes.

3. The article of claim 2, wherein the polymeric material comprises an open cell hydrogel network comprising a plurality of hydrogel nodes crosslinked with one another.

4. The article of claim 2, wherein the hydrogel nodes comprise an average polymer weight percent of from 10 wt % to 80 wt % polymer.

5. The article of claim 2, wherein the hydrogel nodes have an average diameter of from about 10 nm to about 500 μm.

6. The article of claim 2, wherein the open cell network has a characteristic dimension of from about 10 nm to about 1 mm.

7.-8. (canceled)

9. The article of claim 2, wherein at least a portion of the nodes are attached to one another through direct contact or through fibrils.

10. The article of claim 2, wherein the polymeric material has a permeability of from about 1×10⁻¹⁵ m² to about 1×10⁻⁴ m².

11. (canceled)

12. The article of claim 2, wherein the polymeric material comprises from about 20% to about 95% water by mass.

13. The article of claim 2, wherein the polymeric material comprises about 10% polymer by mass to about 30% polymer by mass.

14. The article of claim 2, wherein the polymeric material comprises a modulus of from about 10 kPa to about 100 MPa.

15. (canceled)

16. The article of claim 2, wherein the hydrogel nodes comprise the polymerization product of one or more monomers comprising hydroxyethylmethacrylate (HEMA), a polyethylene glycol (PEG) monomer, a polypropylene oxide (PPO) monomer, lactic acid, glycolic acid, N-isopropylacrylamide (NIPAM), caprolactone, R-3-hydroxybutyrate, vinyl acetate, ethylene, acrylamide, methacrylamide, dimethacrylamide, acrylate, methacrylate, acrylic acid, methacrylic acid, C₂-C₈ alkyl acrylic acids, vinyl ester, vinyl amide, vinyl amine, poly(ethylene glycol) diacrylate (PEGDA), N-vinylpyrrolidone, hydroxyethyl acrylate, hydroxypropyl acrylate, trimethylammonium 2-hydroxypropylmethacrylate, dimethylaminoethyl methacrylate (DMAEMA), dimethylaminoethylmethacrylamide, allyl alcohol, vinylpyridine, glycerol methacrylate, N-(1,1-dimethyl-3-oxobutyl)acrylamide, or any combination thereof.

17. The article of claim 2, wherein the polymeric material comprises an open cell hydrogel network comprising a plurality of hydrogel nodes attached to one another, wherein the hydrogel nodes comprise the polymerization product of hydroxyethylmethacrylate (HEMA), wherein the polymeric material comprises about 10% by mass to about 30% by mass HEMA, and wherein the polymeric material has a permeability of from about 5×10⁻¹² m² to about 1×10⁻⁸ m².

18. The article of claim 17, wherein the polymeric material comprises an open cell hydrogel network comprising a plurality of hydrogel nodes crosslinked with one another, wherein the hydrogel is the polymerization product of hydroxyethylmethacrylate (HEMA) and N,N′-methylenebisacrylamide (BIS).

19.-64. (canceled)

65. A bait formed from the article of claim 2.

66. The bait of claim 65, wherein the bait is formed in the shape of an aquatic animal, an insect, a worm, or fish eggs.

67. The bait of claim 65, further comprising at least one attractant molecule or composition.

68. The bait of claim 67, wherein the attractant molecule comprises inosine, guanylic acid or a salt thereof, inosinic acid or a salt thereof, or any combination thereof.

69. The bait of claim 67, wherein the attractant molecule of or composition comprises a scent molecule, an amino acid, a protein, a peptide, a salt, a carbohydrate, a fish oil, a digested or partially digested aquatic organism, or a combination thereof.

70.-78. (canceled)

79. The bait of claim 67, wherein the attractant molecule or composition is released from the bait over a period of time when the bait is placed in water.

80. The bait of claim 79, wherein the period of time is from about 1 minute to about 10 minutes.

81.-90. (canceled)
